(19) United States
(12) Patent Application Publication
(10) Pub. No.: US 2024/0280364 A1
(43) Pub. Date: Aug. 22, 2024

(54) METHOD FOR DETERMINING PEDOLOGICAL DATA, COMPUTER PROGRAM AND ASSOCIATED DEVICE

(71) Applicants: ATOS France, BEZONS (FR); Institut national de recherche pour l’agriculture, l’alimentation et l’environnement, PARIS (FR)

(72) Inventors: Amine CHEMCHEM, Montpellier (FR); Rida MOUMNI, Montpellier (FR); Paulo PIMENTA, Prades-le-Lez (FR); El Bachir REHHALI, Montpellier (FR); Lalla Aïcha SOW, Montpellier (FR); Philippe LAGACHERIE, Vendémian (FR); Quentin STYC, Saint-Avertin (FR)

(73) Assignees: ATOS France, BEZONS (FR); Institut national de recherche pour l’agriculture, l’alimentation et l’environnement, PARIS (FR)

(21) Appl. No.: 18/444,912

(22) Filed: Feb. 19, 2024

(30) Foreign Application Priority Data
Feb. 22, 2023 (EP) 23305233.1

(51) Int. Cl. G01C 11/04; G01N 33/24

(52) U.S. Cl. CPC G01C 11/04; G01N 33/24

(57) ABSTRACT

The invention relates to a method for determining at least one characteristic of the soil in a geographical zone, the method being implemented by computer and comprising the steps of:
detecting, in at least one georeferenced image (8) associated with the geographical zone, at least one symbol (10) representative of a pedological data measurement point, and an associated unique identifier;
for each symbol (10) detected:
calculating the geographical coordinates of the corresponding measurement point, from the position of said symbol (10) in the image and the georeferencing of the image;
from the corresponding unique identifier, identifying, in a pedological database associated with the geographical zone, the pedological data corresponding to the respective measurement point.

### TECHNICAL FIELD

The present invention relates to a method for determining at least one characteristic of the soil in a geographical zone.

The invention also relates to a computer program and to a device implementing such a method.

The invention applies to the field of pedology, and more particularly to the determination of pedological data in a geographical zone.

### BACKGROUND

To optimize the use of the soil in a given geographical zone (for example to select the crop that will allow an optimal harvest, or to best manage the available water reserves), it is necessary to know the characteristics of the soil at every point of this zone.

Conventionally, to know the characteristics of the soil at a given point, it is known to carry out two complementary analyses at said point.

More precisely, the first analysis consists of carrying out a pedological survey by taking soil cores at regular depth intervals (for example to conduct chemical and physical analyses). Such surveys are easy to carry out in practice.

Additionally, the second analysis consists in cutting a vertical section of the soil (referred to as “soil pit”) to observe the soil profile, that is to say the different strata that make up the soil, from the surface to the bedrock. Such soil pits are dug to a depth of up to more than two meters, which makes their systematic implementation unrealistic.

However, as the characteristics of the soil change little or predictably over time, data relating to former pedological campaigns can be used to estimate the current characteristics of the soil.

However, the use of pedological data relating to former campaigns is not satisfactory.

Indeed, such data are often only available on paper documents, generally of poor quality and in a format that differs from one document to another. As a result, they are processed manually, which is tedious and time-consuming work.

Additionally, these data are generally not georeferenced, making it difficult to obtain a locally accurate estimate of a soil property calculated from these data.

One aim of the present invention is to solve at least one of the shortcomings of the state of the art.

Another aim of the invention is to propose a solution that is automatic, quick and reliable for evaluating the characteristics of the soil at any point in a geographical zone.

### BRIEF SUMMARY

To this end, the invention relates to a method of the above-mentioned type, the method being carried out by computer and comprising the steps of:
detecting, in at least one georeferenced image associated with the geographical zone, at least one symbol representative of a pedological data measurement point, and an associated unique identifier;
for each symbol detected:
   calculating the geographical coordinates of the corresponding measurement point, from the position of said symbol in the image and the georeferencing of the image;
   from the corresponding unique identifier, identifying, in a pedological database (e.g., stored on a non-transitory computer readable storage device) associated with the geographical zone, the pedological data corresponding to the respective measurement point.

Indeed, by virtue of such a process, a unequivocal link is automatically established between the measurement points, the corresponding pedological data and their respective geolocation. This makes it possible to overcome the problem according to which the pedological data from former measurement campaigns were not georeferenced.

This link is established automatically, quickly and reliably, without requiring the intervention of a human operator.

This is particularly advantageous, insofar as knowing the exact geographical coordinates of each measurement point, as well as storing, in digital form, the corresponding pedological data in the database, allows a simulation of new pedological data, this time at any point in the geographical zone, and in a totally automated manner.

Advantageously, the method according to the invention has one or more of the following characteristics, taken individually or in any technically possible combination:
the method further comprises estimating, from the geographical coordinates calculated for each measurement point and the corresponding identified pedological data, pedological data at any point in the geographical zone;
the estimation of pedological data at any point in the geographical zone comprises implementing at least one artificial intelligence model, preferably a fast forest quantile regression;
for each point in the geographical zone, the estimation of pedological data additionally comprises evaluating a corresponding estimation uncertainty;
the evaluation of the estimation uncertainty comprises:
calculating an uncertainty at each measurement point, from a model error between the pedological data estimated at the measurement point and the pedological data associated with said measurement point in the pedological database;
propagating the calculated uncertainty to each point in the geographical zone;
the estimation of pedological data is preceded by an interpolation of the depths between measurement points;
depth interpolation comprises implementing mass-preserving splines;
the estimation of pedological data at any point in the geographical zone is also a function of soil covariables of the geographical zone;
the covariables include information relating to the topography, information relating to the geology and/or information relating to the land use of the geographical zone;
the at least one characteristic of the soil comprises a useful water capacity;
the pedological data comprise upper and lower depths, mass percentages of gravel and stones, a weight percentage of fine soil, a bulk density, an equivalent humidity and/or a structural coefficient for each stratum;
the method comprises, prior to the identification step, a step of populating the pedological database, which includes:
detecting, in each image representative of a survey sheet carried out at a corresponding measurement point of the geographical zone, an identifier of the measurement point and an identifier of the geographical zone;
extracting, from the image, associated pedological data;
generating the unique identifier from the detected identifier of the measurement point and the detected identifier of the geographical zone; and
storing, in the pedological database, pedological data extracted related to the unique identifier of the measurement point;
the extraction of pedological data includes processing that comprises: deleting empty or partially filled lines, correcting soil depth discontinuities, correcting erroneous depths, correcting line offsets and/or deleting duplicates.

According to another aspect of the invention, a computer program is proposed which comprises executable instructions that, when executed by computer, implement the steps of the method as defined hereinbefore.

The computer program can be in any computer language, such as for example machine language, C, C++, JAVA, Python, etc.

According to another aspect of the invention, a device is proposed for determining at least one characteristic of the soil in a geographical zone, the device comprising a database and a processing unit,
the database being configured to store pedological data associated with the geographical zone, the processing unit being configured to:
detect, in at least one georeferenced image associated with the geographical zone, at least one symbol representative of a pedological data measurement point, and an associated unique identifier;
for each symbol detected:
   calculate the geographical coordinates of the corresponding measurement point, from the position of said symbol in the image and the georeferencing of the image;
   from the corresponding unique identifier, identify, in the database, the pedological data corresponding to the respective measurement point.

The device according to the invention can be any type of device such as a server, a computer, a tablet, a calculator, a processor, a computer chip, programmed to implement the method according to the invention, for example by executing the computer program according to the invention.

### BRIEF DESCRIPTION OF THE FIGURES

The invention will be better understood on reading the following description, given solely by way of non-limiting example and with reference to the accompanying drawings, wherein:

FIG. 1 is a schematic depiction of a device according to the invention; and

FIG. 2 is an example of a georeferenced image provided as input to the device of FIG. 1.

### DETAILED DESCRIPTION

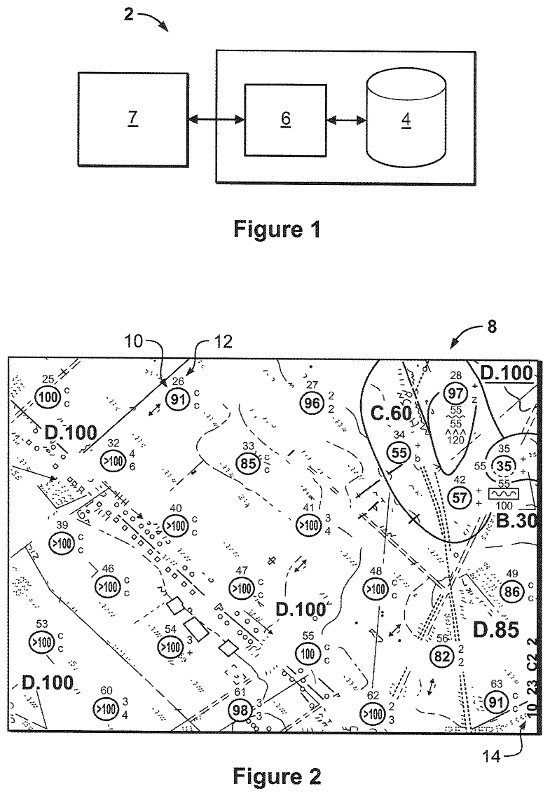

A device 2 according to the invention is schematically illustrated in FIG. 1.

The device 2 is intended to determine at least one characteristic of the soil in one or more given geographical zone(s).

For example, one such characteristic is a useful water capacity.

The device 2 includes a database 4 (also referred to as “memory”) and a processing unit 6 which are linked together. Preferably, the device 2 also includes a display 7 connected to the processing unit 6.

The device 2 may be in hardware form, such as a computer, a server, a processor, an electronic chip, etc. Alternatively, or additionally, the device 2 may be in software form, such as a computer program, or an application, for example an application for a user device such as a tablet or smartphone.

### Memory 4

The memory 4 is configured to store pedological data associated with each geographical zone.

More precisely, the memory 4 is configured to store pedological data associated with at least one measurement point of the geographical zone.

“Measurement point” is intended to mean, within the meaning of the present invention, a point in the geographical zone where pedological data have been collected, in particular by carrying out a soil pit or a pedological survey (taking soil cores at successive depths).

For example, such pedological data comprise, for each stratum of the soil (also referred to as “soil horizon”), upper and lower depths, mass percentages of gravel and stones, a weight percentage of fine earth, an apparent density, an equivalent humidity and/or a structural coefficient.

In the memory 4, each measurement point is identified by virtue of a corresponding unique identifier. Additionally, each unique identifier is matched, in the memory 4, with the pedological data recorded at the associated measurement point.

Preferably, the memory 4 is also configured to store soil covariables relating to the geographical zone.

Conventionally, such covariables include information relating to the topography, information relating to the geology and/or information relating to the land use of the geographical zone.

### Processing Unit 6

The processing unit 6 is a hardware processing unit, such as a processor, an electronic chip, a calculator, a computer, a server, etc. Alternatively, or additionally, the processing unit 6 is a software processing unit, such as an application, a computer program, a virtual machine, etc.

The processing unit 6 is intended to locate the measurement points and to identify the corresponding pedological data.

Advantageously, the processing unit 6 is also configured to estimate pedological data at any point in the geographical zone from the pedological data at the measurement points. This is advantageous, insofar as knowing the pedological data at any point makes it possible to determine, at any point in the geographical zone, the characteristics of the soil.

Preferably, the processing unit 6 is also configured to, beforehand, populate the database 4, that is to say, store, in the database 4, the pedological data recorded at the measurement points.

### Populating the Database

To populate the memory 4, the processing unit 6 is configured to receive at least one image representative of pedological data recorded at a measurement point of the geographical zone.

For example, such an image results from the digitization of a survey sheet relating to a pedological survey carried out at a given measurement point.

Alternatively, or in a complementary fashion, such an image results from the digitization of a soil profile relating to a soil pit carried out at a given measurement point.

The processing unit 6 is also configured to detect, in each image received, an identifier of the measurement point and an identifier of the geographical zone. In particular, to carry out such a detection, the processing unit 6 is configured to implement character recognition software, for example configured to implement an artificial intelligence model.

Advantageously, such a detection is based, at least in part, on prior knowledge of the images. For example, the processing unit 6 is configured to search for the identifiers of the measurement point and the geographical zone in a specific part of the image which is known to be likely to contain said identifiers. This is advantageous, insofar as such a configuration reduces image processing time and increases the reliability thereof.

The processing unit 6 is also configured to extract the pedological data from the image. In particular, to carry out such an extraction, the processing unit 6 is configured to implement character recognition software, for example configured to implement an artificial intelligence model.

Advantageously, such a detection is based, at least in part, on prior knowledge of the images. For example, the processing unit 6 is configured to search for each of the pedological data in a specific part of the image which is known to be likely to contain said data. This is advantageous, insofar as such a configuration reduces image processing time and increases the reliability thereof.

Advantageously, the processing unit 6 is, additionally, configured to implement, during such an extraction, a processing of the extracted data. Such processing comprises deleting empty or partially filled lines, correcting soil depth discontinuities, correcting erroneous depths, correcting line offsets and/or deleting duplicates. This operation is advantageous, insofar as it leads to a harmonization of the extracted data and/or to the deletion of incorrect data, which results in a reduction in the number of erroneous extracted data on which the estimation of the pedological data in the geographical zone would be likely to be based.

The processing unit 6 is also configured to generate, for each measurement point, the unique identifier mentioned previously. More precisely, for each measurement point, the processing unit 6 is configured to generate the unique identifier from the identifier of the measurement point and the identifier of the geographical zone detected. For example, the unique identifier is the result of the concatenation of the identifier of the measurement point and the identifier of the geographical zone.

Additionally, for each measurement point, the processing unit 6 is configured to store, in the memory 4, the corresponding extracted pedological data related to the respective unique identifier.

At the end of this process, the database 4 stores, for each measurement point, the corresponding pedological data, related to the respective unique identifier.

### Determining the Geographical Coordinates of Each Measurement Point

The processing unit 6 is also configured to match the pedological data stored in the memory 4 to geographical coordinates. More precisely, the processing unit 6 is configured to carry out such matching on the basis of at least one georeferenced image associated with the geographical zone and said pedological data stored in the memory 4. In this case, each georeferenced image forms, for example, the smallest georeferenced square (also referred to as “mapillon”) of a projected coordinate system of an area in which pedological data has been collected.

Such a georeferenced image is an image associated with the geographical zone (for example an image representative of at least part of the geographical zone) and in which markers (also referred to as “symbols”) associated with the measurement points have been placed.

“Georeferenced image” is intended to mean, within the meaning of the present invention, an image for which at least three points have known geographical coordinates.

One example of such a georeferenced image 8 is visible in FIG. 2.

As appears in FIG. 2, each symbol 10 (herein, a circle) is arranged, in the georeferenced image 8, at a position which corresponds to the actual position of the corresponding measurement point in the geographical zone.

Additionally, each symbol 10 is associated with an identifier 12 of the measurement point. In the georeferenced image 8, the identifier 12 of the measurement point takes the form of a two-digit number located above the corresponding symbol 10.

The georeferenced image 8 further includes an identifier 14 of the geographical zone. In the georeferenced image 8, the identifier 14 of the geographical zone depicted, located in the lower right corner, is: 10_23_C2_2.

In this case, the processing unit 6 is configured to detect, in each georeferenced image, each symbol 10 representative of a measurement point. For example, to carry out such a detection, the processing unit 6 is configured to implement a shape recognition algorithm.

Additionally, for each symbol 10, the processing unit 6 is configured to detect the corresponding identifier 12. For example, the processing unit 6 is configured to search for the identifier 12 in a part of the georeferenced image 8 selected relative to the position of the symbol 10 in said image 8, and which is known to be likely to contain the identifier 12.

The processing unit 6 is also configured to detect the identifier 14 of the geographical zone. For example, the processing unit 6 is configured to search for the identifier 14 in a part of the georeferenced image 8 which is known to be likely to contain the identifier 14.

Preferably, to detect the identifiers 12, 14, the processing unit 6 is configured to implement character recognition software.

Additionally, for each measurement point associated with a symbol 10 detected, the processing unit 6 is configured to determine the unique identifier from the identifier 12 of the measurement point and the identifier 14 of the geographical zone.

For each symbol 10 detected, the processing unit 6 is also configured to calculate the geographical coordinates of the corresponding measurement point, from the position of the symbol 10 in the georeferenced image 8 and from the georeferencing of said image 8. For example, for each symbol 10 detected, the processing unit 6 is configured to calculate the geographical coordinates of the corresponding measurement point from the position of the barycenter of the symbol 10 in the georeferenced image 8.

Additionally, for each measurement point, the processing unit 6 is configured to identify, in the memory 4, corresponding pedological data.

More precisely, the processing unit 6 is configured to determine the unique identifier from the identifier 12 of the measurement point and the identifier 14 of the geographical zone.

The processing unit 6 is configured to then load, from the memory 4, and for each measurement point, the pedological data associated with the determined unique identifier associated with it.

Preferably, the processing unit 6 is also configured to store, from the memory 4, and for each measurement point, the corresponding determined geographical coordinates.

### Calculating Pedological Data in the Geographical Zone

Preferably, the processing unit 6 is also configured to estimate pedological data at any point in the geographical zone, based on the geographical coordinates calculated for each measurement point and the corresponding identified pedological data.

For example, to carry out such an estimation, the processing unit 6 is configured to implement a previously trained artificial intelligence model, preferably a fast forest quantile regression. More preferably, the processing unit 6 is configured to carry out such an estimation by implementing, for each soil horizon, a respective artificial intelligence model.

Advantageously, the processing unit 6 is configured to carry out this estimation on the basis of the soil covariables of the geographical zone stored in the memory 4. For example, in the case of using an artificial intelligence model to estimate the pedological data, the model is also configured to take said soil covariables as input in order to carry out said estimation.

Preferably, to carry out such an estimation, the processing unit 6 is configured to first implement an interpolation of the depths between the measurement points. In this fashion, the depth of the pedological data associated with the different measurement points is harmonized.

For example, the processing unit 6 is configured to carry out such an interpolation at several depths, such as 30 cm, 60 cm, 100 cm and 200 cm. In this fashion, a plurality of soil horizons is defined, each being comprised between two successive depths.

In particular, the processing unit 6 is configured to implement mass-preserving splines in order to carry out such an interpolation. Alternatively, to carry out such an interpolation, the processing unit 6 is configured to implement any other suitable interpolation method, such as inverse distance weighting or even kriging.

Advantageously, for each point in the geographical zone, in addition to the corresponding pedological data, the processing unit 6 is also configured to evaluate a corresponding estimation uncertainty.

More preferably, for each point in the geographical zone, such an uncertainty evaluation is carried out for each soil horizon.

For example, in the case where an artificial intelligence model is trained to estimate the pedological data at every point in the geographical zone, cross-validation (the implementation of which is known to a skilled person) is implemented when training the model on the basis of pedological data (possibly after interpolation) associated with the measurement points. This leads to determining an uncertainty (that is to say, a reliability) of the estimation of pedological data by the model, at each measurement point. Preferably, and as indicated previously, such an uncertainty is evaluated for each soil horizon.

Alternatively, the processing unit 6 is configured to implement any other method of calculating uncertainty on the estimation at each measurement point, which is a function of an error of the model between, on the one hand, the pedological data estimated at the measurement point and, on the other hand, the pedological data actually measured (and thus associated with said measurement point in the memory 4).

In particular, to carry out such an uncertainty evaluation, the processing unit 6 is configured to propagate, to each point of the geographical zone, the uncertainty calculated at the measurement points. Preferably, such a propagation is carried out separately for each soil horizon.

The processing unit 6 is also configured to calculate at least one characteristic of the soil at any point in the geographical zone from the estimated pedological data. In this fashion, for each geographical zone, a map of the spatial variations of each characteristic of the soil is obtained, as well as, preferably, a map of the associated uncertainties.

For example, the processing unit 6 is configured to calculate a useful water capacity in the soil, for example by implementing the formula:

$$R U = {\sum }_{i} d {h}_{i} b {d}_{i} ( \frac{100 - s {t}_{i}}{100} ) {b}_{i} {H}_{eqi}$$

wherein RU is the useful water capacity at a given point in the geographical zone,
H_{eqi} is the equivalent humidity of the soil horizon i at said point,
bᵢ is the texture coefficient of the soil horizon i at said point,
stᵢ is the volume content of coarse elements (that is to say, elements having a diameter greater than 2 cm) of the soil horizon i at said point,
bdᵢ is the apparent density of the fine earth of the soil horizon i at said point, and
dhᵢ is the thickness of the soil horizon i at said point.

Preferably, for each geographical zone, the processing unit 6 is also configured to transmit, to the display 7, the corresponding map of the spatial variations of each characteristic of the soil to be displayed, and, where appropriate, the map of the associated uncertainties.

The use of a fast forest quantile regression is advantageous, the inventors having noted the surprising effect according to which such an artificial intelligence model offers very satisfactory performance for the present application.

The estimation error made by the artificial intelligence model at the measurement points is likely to be easily obtained. Consequently, the use of a propagation of said estimation error is advantageous, insofar as it allows a determination of the uncertainty at any point in the geographical zone which requires little calculation time.

The use of an interpolation of the depths between the measurement points is advantageous, insofar as it leads to harmonization of the depths of the soil horizons, which are likely to vary greatly from one measurement point to another. In this fashion, the estimation of pedological data is carried out for new fictitious horizons, of fixed depth.

The implementation of mass-preserving splines is advantageous, insofar as the differences between the pedological data before and after interpolation are minimal. This results from the fact that the average of the values for the fictitious horizons generated and the initial value introduced to generate them are equal.

The use of soil covariables for the estimation of pedological data over the entire geographical zone is advantageous, insofar as it increases the reliability of the estimation by taking into account the real nature of the terrain.

Determining the useful water capacity is advantageous, insofar as knowledge of such a characteristic is useful for optimizing the use of the soil, for example to select the crop that will allow a satisfactory harvest, or even to enable better management of the available water reserves.

The estimation of the mass percentages of gravel/stones, the weight percentage of fine earth, the apparent density, the equivalent humidity and/or the structural coefficient is advantageous, insofar as it allows a more reliable estimation of the useful water capacity.

### Operation

The operation of the device 2 will now be described.

During an initialization step, the processing unit 6 populates the memory 4. More precisely, the processing unit 6 receives one or more image(s) each representative of pedological data recorded at a measurement point of the geographical zone.

Then, the processing unit 6 detects, in each image received, the identifier of the measurement point and the identifier of the geographical zone.

The processing unit 6 also extracts the pedological data from the image. Advantageously, during this extraction, the processing unit 6 implements a processing of the extracted data to reduce the number of erroneous data.

The processing unit 6 also generates, for each measurement point, the unique identifier mentioned previously, in particular the identifier of the measurement point and the identifier of the geographical zone detected.

Then, for each measurement point, the processing unit 6 stores, in the memory 4, the corresponding extracted pedological data related to the respective unique identifier.

Then, during a localization step, at least one georeferenced image is provided as input to the processing unit 6.

In this case, the processing unit 6 detects each symbol 10 representative of a measurement point, the corresponding identifier 12, as well as the identifier 14 of the geographical zone.

Then, for each symbol 10 detected, the processing unit 6 calculates the geographical coordinates of the corresponding measurement point, and determines the unique identifier of the corresponding measurement point.

Then, for each measurement point, the processing unit 6 identifies, in the memory 4, the corresponding pedological data by means of the unique identifier determined, and loads them.

Then, during a calculation step, the processing unit 6 estimates the pedological data at any point in the geographical zone, from the geographical coordinates calculated for each measurement point and the corresponding identified pedological data.

Advantageously, for each point in the geographical zone, the processing unit 6 also evaluates a corresponding estimation uncertainty.

Then, the processing unit 6 calculates each characteristic of the soil from the estimated pedological data.

Then, preferably, the processing unit 6 transmits, to the display 7, the map of the spatial variations of each characteristic of the soil (and, where applicable, the map of the associated uncertainties) to be displayed.

1. A method for determining at least one characteristic of soil in a geographical zone, the method being implemented by computer and comprising:
detecting, in at least one georeferenced image associated with the geographical zone, at least one symbol representative of a pedological data measurement point, and an associated unique identifier; and
for each symbol detected:
calculating geographical coordinates of a corresponding measurement point, from a position of said symbol in the image and the georeferencing of the image; and
from the corresponding unique identifier, identifying, in a pedological database associated with the geographical zone, the pedological data corresponding to the respective measurement point.

2. The method according to claim 1, further comprising estimating, from the geographical coordinates calculated for each measurement point and the corresponding identified pedological data, the pedological data at any point in the geographical zone.

3. The method according to claim 2, wherein, for each point in the geographical zone, the estimation of the pedological data further comprises an evaluation of a corresponding estimation uncertainty.

4. The method according to claim 2, wherein the estimation of pedological data at any point in the geographical zone comprises implementing at least one artificial intelligence model, preferably a fast forest quantile regression.

5. The method according to claim 4, wherein, for each point in the geographical zone, the estimation of the pedological data further comprises an evaluation of a corresponding estimation uncertainty.

6. The method according to claim 5, wherein the evaluation of the estimation uncertainty comprises:
calculating an uncertainty at each measurement point, from a model error between the pedological data estimated at the measurement point and the pedological data associated with said measurement point in the pedological database; and
propagating the calculated uncertainty to each point in the geographical zone.

7. The method according to claim 2, wherein the estimation of pedological data is preceded by an interpolation of depths between measurement points.

8. The method according to claim 7, wherein the depth interpolation comprises implementing mass-preserving splines.

9. The method according to claim 2, wherein the estimation of pedological data at any point in the geographical zone is also a function of soil covariables of the geographical zone.

10. The method according to claim 9, wherein the covariables include information relating to topography, information relating to geology and/or information relating to land use of the geographical zone.

11. The method according to claim 1, wherein the at least one characteristic of the soil comprises a useful water capacity.

12. The method according to claim 1, wherein the pedological data comprises upper and lower depths, mass percentages of gravel and stones, a weight percentage of fine earth, an apparent density, an equivalent humidity and/or a structural coefficient for each stratum.

13. The method according to claim 1, comprising, prior to identifying the pedological data, populating the pedological database by performing steps including:
detecting, in each image representative of a survey sheet carried out at a corresponding measurement point of the geographical zone, an identifier of the measurement point and an identifier of the geographical zone;
extracting, from the image, associated pedological data;
generating the unique identifier from the detected identifier of the measurement point and the detected identifier of the geographical zone; and
storing, in the pedological database, pedological data extracted related to the unique identifier of the measurement point.

14. The method according to claim 13, wherein the extraction of pedological data includes processing that comprises deleting empty or partially filled lines, correcting soil depth discontinuities, correcting erroneous depths, correcting line offsets and/or deleting duplicates.

15. A computer program comprising executable instructions which, when they are executed by computer, implement a method according to claim 1.

16. A device for determining at least one characteristic of soil in a geographical zone, the device comprising a database and a processing unit,
the database being configured to store pedological data associated with the geographical zone,
the processing unit being configured to:
detect, in at least one georeferenced image associated with the geographical zone, at least one symbol representative of a pedological data measurement point, and an associated unique identifier; and
for each symbol detected:
calculate geographical coordinates of a corresponding measurement point, from a position of said symbol in the image and the georeferencing of the image; and
from the corresponding unique identifier, identify, in the database, the pedological data corresponding to the respective measurement point.
